(19) United States
(12) Patent Application Publication
(10) Pub. No.: US 2021/0137400 A1
(43) Pub. Date: May 13, 2021

(54) Optically Excited Biopotential Phantom

(71) Applicant: X Development LLC, Mountain View, CA (US)

(72) Inventor: Russell Mirov, Los Altos, CA (US)

(21) Appl. No.: 16/682,570

(22) Filed: Nov. 13, 2019

(51) Int. Cl. A61B 5/0402; A61B 5/04; A61B 5/0476; A61B 5/0488

(52) U.S. Cl. CPC A61B 5/04021; A61B 5/04008; A61B 2017/00707; A61B 5/0488; A61B 5/0476

(57) ABSTRACT

The technology provides a system and method for simulating and detecting bio signals such as brain bio-signals. The technology can be used for medical or non-medical purposes, for instance to simulate or evaluate certain medical conditions using a physical brain-type phantom body. A set of optical fibers provides modulated signals received from an optical signal modulator, which is managed by a controller to generate repeatable signals with high fidelity. The modulated signals are received by a set of emission elements such as photoreceivers or other optical electrodes disposed within or otherwise about the phantom body. The emission elements output electrical signals corresponding to the input modulated optical signals. The electrical signals are detected by a set of sensors. The sensors are coupled to a receiver device that is able to evaluate the electrical signals, such as for an electroencephalograph (EEG), electrocardiogram (ECG), electromyogram (EMG) or magnetoencephalography (MEG) diagnostic system.

### CROSS-REFERENCE TO RELATED APPLICATION

The present application is related to U.S. application Ser. No. 16/682,621, entitled Opto-Electronic Biopotential Controller, Attorney Docket No. ATOZX 3.0F-2053 [9019], filed concurrently herewith, the entire disclosure of which is incorporated by reference herein.

### BACKGROUND

Physical bio-phantoms have been used to simulate various types of human tissue, including the brain. In this type of system, a set of electrodes may be placed at various points along the phantom by running wires through it. The electrodes are actuated with electrical pulses. However, the placement and amount of wiring may create interference (e.g., crosstalk) that can adversely impact signal quality and prevent accurate signal evaluation. For instance, magnetoencephalography (MEG) may be significantly affected by electrical crosstalk.

### BRIEF SUMMARY

The technology relates to a system for simulating bio signals such as brain bio-signals, as well as detection and evaluation of such signals. This system can be used for medical or non-medical purposes, e.g., to simulate or evaluate certain medical conditions, to provide a brain control interface (BCI) for a computer application, etc. As discussed further herein, a physical brain-type phantom is employed that is configured to simulate various biopotentials in a repeatable and calibrated manner. Furthermore, the phantom structure does not need to be homogenous. Rather, it may have different layers or regions to mimic the skull, brain, skin, hair, etc.

According to one aspect, an optically excited biopotential phantom system is provided that includes a phantom body structure. The phantom body structure has an electrically conductive bulk material. The phantom body structure is configured to represent one or more body tissues or structures. The system also includes a set of optical fibers. A first section of each optical fiber is received within the phantom body structure and a second section of each optical fiber extends from the phantom body structure. The first section has a first end of a respective one of the set of optical fibers and the second section has a second end of the respective optical fiber. The system also includes a set of optodes configured to operate in a photoresponsive mode. Each one of the set of optodes is optically coupled to a corresponding one of the set of optical fibers. The second end of each one of the set of optical fibers is configured to receive an optical waveform from an optical modulation module and to pass the received optical waveform to the first end thereof. The first end of each one of the set of optical fibers is configured to excite a corresponding one of the set of optodes optically coupled thereto based on the received optical waveform. And each optode is configured to generate an electrical signal based on an excitation response to the received optical waveform. The generated electrical signals from the set of optodes are detectable by one or more sensors disposed on a surface of the phantom body structure.

The set of optodes may be arranged in a 2D or a 3D pattern within the phantom. In one scenario, a 3D pattern includes at least one densely populated region and at least one sparsely populated region having fewer optodes than the at least one densely populated region. Each optode of the set of optodes may be either a photodiode or an LED configured to operate as a photoreceiver.

Each optode may be optically coupled to the corresponding optical fiber with an adhesive. The adhesive may be an optically clear adhesive. The adhesive may include a phosphorous material. The phantom body structure may include a scaffolding holding the set of optical fibers in a 3D pattern within the phantom. The bulk material can be a gel, a liquid or a solid. In addition, each one of the set of optical fibers may be either a single mode fiber or a multi-mode fiber.

In one example, the system further comprises the optical modulation module. In addition or alternatively, the system may further comprise the one or more sensors. The system may also include a receiver device operatively coupled to the one or more sensors. For instance, the one or more sensors may be, e.g., electroencephalograph (EEG), electrocardiogram (ECG), electromyogram (EMG) or magnetoencephalography (MEG) sensors. The bulk material can include electrically conductive salt ions or other electrically conductive materials. The photoresponsive mode may be a photovoltaic mode.

According to another aspect of the technology, a method of operating an optically excited biopotential phantom system is provided. The system including a phantom body structure having a set of optodes arranged therein. The method comprises selecting, by one or more processors of a control module in response to an input, a test or condition of interest from a set of biopotential scenarios; the one or more processors causing a signal modulator to modulate light emitted from one or more light sources according to the selected test or condition of interest from the set of biopotential scenarios; and the modulated light causing selected ones of the set of optodes to operate in a photoresponsive mode so that each selected optode outputs an electrical signal as an excitation response to the modulated light, whereby the electrical signal output from each selected optode is detectable by one or more sensors disposed on a portion of the phantom body structure.

In one example, the method further comprises detecting, by the one or more sensors disposed on the portion of the phantom body structure, at least some of the output electrical signals; and evaluating, by a receiver device operatively coupled to the one or more sensors, the detected electrical signals. Evaluating the detected electrical signals may include determining that the detected electrical signals correspond to a particular test or condition. The method may also include comparing the particular test or condition to the selected test or condition of interest to determine whether the receiver device correctly evaluated the selected test or condition of interest.

The selected test or condition of interest may correspond to a brainwave pattern, a medical condition, a stimuli to be evaluated, or a brain control interface command. The method may also include calibrating the set of optodes and/or the one or more sensors.

And according to yet another aspect, a method of fabricating an optically excited biopotential phantom system is provided. The method comprising: providing a scaffolding corresponding to a biological structure or structure; coupling a set of optodes to a set of optical fibers, each optode of the set being optically engaged with a first end of a corresponding one of the optical fibers, each optical fiber in the set being configured to pass modulated light from a second end thereof to the first end, and each optode of the set of optodes being configured to operate in a photoresponsive mode so that each optode outputs an electrical signal as an excitation response to the modulated light; arranging the first ends of the set of optical fibers along the scaffolding so that the set of optodes are placed in a predetermined 2D or 3D pattern; and forming a phantom body structure, the phantom body structure comprising a weakly conductive bulk material.

The bulk material may be a liquid, a gel or a solid. The weakly conductive bulk material may include salt ions or other charged particles.

Coupling the set of optodes to the set of optical fibers may include securing each corresponding optode to the first end of the corresponding optical fiber with an adhesive. The adhesive may be an optically clear adhesive. The adhesive may include a phosphorous material.

Forming the phantom body structure may include pouring the bulk material around the scaffolding. And providing the scaffolding may include 3D printing the scaffolding.

### BRIEF DESCRIPTION OF THE DRAWINGS

FIG. 1 illustrates an example brain to emulate in accordance with aspects of the technology.

FIGS. 2A-B illustrate an example bio-phantom system in accordance with aspects of the technology.

FIG. 3 illustrates an example optical fiber-photoreceiver coupling in accordance with aspects of the technology.

FIGS. 4A-B illustrate light pulse responses in accordance with aspects of the technology.

FIG. 5 illustrates an example method of operation in accordance with aspects of the technology.

FIG. 6 illustrates an example method of fabrication in accordance with aspects of the technology.

### DETAILED DESCRIPTION

FIG. 1 illustrates one type of biological tissue 100, in particular a brain, that can be emulated according to the technology disclosed herein. The human brain is a soft tissue structure that includes billions of neurons and many more synaptic connections disposed throughout a complex 3D framework. As noted above, a bio-phantom can be used to simulate certain brain activity. However, arranging the wiring and placement of electrodes in a bio-phantom may be challenging, even for a small amount of electrodes. Furthermore, crosstalk or other interference can corrupt signals, rendering them unsuitable for medical or non-medical purposes (e.g., diagnostics, BCI). The following provides a bio-phantom architecture and emulation system that minimizes such problems and enables robust testing and analysis. For instance, bio-phantoms as discussed herein can be used to create electrical signals that are detectable by various types of sensors used for symptom evaluation, analytics or other purposes.

### Example Implementation

FIG. 2A illustrates an example bio-phantom system 200 including a phantom body structure 202, a set of optical fibers 204, and a corresponding set of emission elements such as photoreceivers or other optical electrodes (“optodes”) 206 disposed within or otherwise about the phantom body structure 202. The system 200 also includes a set of sensors 208, which are coupled to a receiver device 210, as well as an optical modulation and control module 212 that is coupled to the set of optical fibers 204.

FIG. 2B illustrates a functional view 250 of the example bio-phantom system of FIG. 2A. The receiver device 210 may be a diagnostic or computing system. By way of example, diagnostic systems such as electroencephalograph (EEG), electrocardiogram (ECG), electromyogram (EMG) or MEG systems can be employed. Alternatively, the receiver device 210 may be part of or coupled to a BCI system.

As shown, the optical modulation and control module 212 includes a controller 252 having one or more processors 254 and memory 256, as well as an optical signal modulator 258. Memory 256 stores instructions and data that may be executed or otherwise used by the processor(s) 254. For instance, the memory may store information regarding different biopotential scenarios or other tests to be conducted, excitation patterns to be generated, and/or conditions to be evaluated by the system. The one or more processors 254 may be, e.g., a controller or CPU. Alternatively, the one or more processors 254 may be a dedicated device such as an ASIC, DSP, FPGA or other hardware-based device. The memory 256 may be of any type capable of storing information accessible by the processor(s) in a non-transitory manner, such as solid state flash memory, hard disc, optical medium or the like.

The instructions may be any set of instructions to be executed directly (such as machine code) or indirectly (such as scripts) by the processor(s). For example, the instructions may be stored as computing device code in the non-transitory memory. In that regard, the terms “instructions” and “programs” may be used interchangeably herein. The instructions may be stored in object code format for direct processing by the processor(s), or in any other computing device language including scripts or collections of independent source code modules that are interpreted on demand or compiled in advance. The data may be retrieved, stored or modified by one or more processors in accordance with the instructions. As an example, the data may comprise one or more modulation schemes to be used in a training system, where the modulation schemes are associated with one or more brainwave patterns, medical conditions or stimuli to be evaluated by the training system.

The signal modulator 258 is configured to set or modulate one or more light patterns that are to be propagated along the set of optical fibers from one or more light sources 260. The light source(s) 260 may be incorporated into the module 212 or be a separate source. By way of example, the light source 260 may comprise one or more LEDs arranged in a linear array or 2D matrix. This optical modulation approach can provide effectively arbitrarily high bandwidth, which can permit finely tuned waveform patterns. Here, light output from the light source 260 may be time multiplexed across some or all of the fibers in the set.

The light patterns can be applied to one, some or all of the optical fibers in the set. As shown by arrow 262, the light patterns are received by the phantom body structure 202. As described in more detail below, the optodes generate electrical signals in response to the received light patterns, and those electrical signals are detected by the sensors. As shown by arrow 264, the detected signals are passed to the receiver device 210 for processing. And as indicated by dashed arrow 266, the optical modulation and control module 212 and the receiver device 210 may be in direct or indirect communication with one another. This may be done, for instance, to provide feedback to the module 212 as part of a machine learning or other training operation. Alternatively or additionally, this could be used as part of the phantom device manufacturing process to calibrate or normalize each channel's performance and thus account for variations in optical source, fiber, phosphor, glue, silicon detector performance/efficiency, and actual position of the optode within the phantom.

The signal modulator 258 is able to generate repeatable optical signals in response to instructions from the controller 252. Such optical signals can include one or more patterns for emulating selected types of brain activity. In particular, modulation of the light source(s) will result in different waveform patterns. The controller 252 may be programmed with waveform patterns to simulate normal and pathological conditions. As a result, the optical signals generated by the signal modulator and propagated through the set of optical fibers will cause the optodes to emit electrical signals in different patterns corresponding to the selected brain activity. By way of example, the brain activity may include, e.g., Alpha, Delta, Theta, and Beta waves. “Sleep Spindles”, slow-waves, and different types of seizures.

This arrangement allows for a repeatable approach that can be used for testing of different conditions and scenarios. For instance, a test or condition may be selected, and a particular optical pattern applied to some or all of the optodes corresponding to that test or condition. Resultant electrical signals output by some or all of the optodes may be evaluated by a received device (e.g., an EEG, ECG, EMG or MEG device), for example to try and identify a particular test or condition. The evaluation by the receiver device may then be compared to the selected test or condition to determine whether the receiver device performed the evaluation correctly (e.g., within a threshold accuracy of 85-95%, or more or less). The arrangement also enables calibration of the sensors and the receiver device. In particular, because the optical approach avoids crosstalk or other interference, the system can repeatedly produce the same waveform patterns with high fidelity. This allows one to develop and calibrate equipment that is particularly beneficial in research and clinical settings. In another approach, machine learning techniques can be employed with the system, for instance to help identify particular medical conditions, or to associate particular brain activity with instructions used to control a computing device or other component as part of a BCI system.

### Example Implementation and Operation

According to one aspect of the technology, the set of optical fibers 204 is at least partly arranged within the phantom body structure 202. A first end of each optical fiber is coupled to the optical modulation and control module 212, while an opposing second end is disposed within the phantom body structure 202 remote from the module 212. Each fiber is configured to convey a modulated light signal to a particular location along or otherwise within the phantom body structure. The modulation can be accomplished in various ways by the optical modulation and control module. This can include varying the intensity, pulse width, pulse duration, polarization and/or color, etc. of the propagated light.

The second end of each fiber is coupled to an optode. Each optode may be, e.g., a photodiode or light-emitting diode (LED) such as a surface mount LED. FIG. 3 illustrates an enlarged view of dashed region 300 of FIG. 2A. As shown in this view by dashed line 302, a modulated optical signal is propagated along optical fiber 304 towards optode 306. While in one example the optode 306 may be directly connected to the optical fiber 304, in another example optical coupling may be accomplished using an optically clear adhesive 308 or other coupling mechanism.

Significant advantages to this approach are that little to no detectable signal loss occurs along the fiber, and there is effectively no crosstalk between the fibers, either leading into the phantom body structure or within the phantom body structure itself. Avoidance of such signal degradation or interference makes the system operation repeatable and robust. One benefit of this is the ability to use fibers of any needed length, which is particularly helpful when the phantom body structure is located in a shielded enclosure (e.g., for MEG testing) and the optical modulator and/or overall control system is located remote from or otherwise outside of the shielded enclosure. For instance, each fiber may be, e.g., 1 m, 10 m, 100 m in length, or more or less. The optical fibers may be single mode or multi-mode.

In one scenario using an optically clear adhesive, the adhesive may be applied and cured using a UV light source. Here, a phosphor slurry or other additive, as shown by speckled elements 310, the may be incorporated with the adhesive to extend (stretch) the time each light pulse is incident on the optode. This may be done to avoid a flicker effect at the optode. According to one aspect, the phosphor slurry or other additive may be particularly beneficial in instances where the optical modulation involves one or more sets of brief light pulses across a series of fibers. By way of example, rapid pulsing of the light source for one or more fibers would result in rapid electrical pulses appearing at the corresponding optode in the phantom body structure.

Use of a phosphor slurry could modify this to result in a continuous fading trail (“glow”) rather than discrete pulses. FIGS. 4A-B illustrate an example of a fading trail in response to a series of light pulses. In particular, FIG. 4A illustrates a view 400 of a series of light pulses of different intensities at times t₀, t₁, . . . t₆, which may be generated by the signal modulator as directed by the controller. By way of example, each pulse may last less than 10 ms, for instance 0.1-5.0 ms, or more or less. The time between pulses may be on the order of 10-50 ms, or more or less, and the time between pulses may vary.

In this example, while the view 400 shows the pulses as occurring with uniform spacing in time, this need not be the case. Rather, adjacent pulses may occur more quickly or slowly depending on the type(s) of signals the system is emulating, and the type(s) of resultant electrical patterns desired. By way of example only, the time between t₀ and t₁ may be 10 ms, while the time between t₁ and t₂ may be 20 ms. The intensity may vary on an absolute or relative scale, for instance, from 1% to 100% of maximum intensity. FIG. 4B illustrates another view 410, in which the slurry provides a persistent, fading light instead of instantaneous pulses, thereby prolonging the time the light is visible to the sensors. Thus, in this scenario, one or more fibers can receive a very narrow pulse of high intensity, where the phosphor is able to stretch out the pulse to remove flicker.

The choice of source light wavelength may be based on the sensitivity of the detector(s) and the availability of the source. The phosphor type and slurry composition may also be varied. For instance, a particular arrangement may be selected to absorb energy at a particular wavelength and re-emit at another (fluoresce). According to one scenario, if the goal is to emulate neural spiking in a human brain, then either no or a short-lifetime phosphor can be used. If slower frequency waveforms are desired, then a slower or longer lifetime phosphor can be employed as a low-pass filter or optical signal integrator.

The optodes can be operated in a photovoltaic mode to produce a photoelectric effect. For instance, the anode and cathode pads 312, 314 of each optode may be left exposed as shown in FIG. 3. These pads may be gold plated, and are thus relatively inert and highly conductive. As photons are emitted from the fiber and are received at the semiconductor junction of the optode, a potential (e.g., up to 1.5 volts or more or less) is developed across the anode/cathode junction. This, in turn, creates an electrical signal that is detectable, for instance, by an EEG, ECG, EMG, MEG or BCI sensor. The potential's amplitude is related to the intensity of the emitted light, in accordance with the modulation of the light. Thus, the controller may finely tune the electrical signals to be detected by the sensors by manipulating the emitted light intensity from the signal modulator.

A radiating element may act as a mechanism for getting more surface area of the optode in contact with the conductive substrate material of the phantom body structure. This may be of particular interest in situations where there is a low-density optode arrangement within the structure so that each optode needs to handle a larger area or volume. In very high density situations it may be more desirable for each fiber/detector endpoint to be as small as possible, so that the sensors are not receiving electrical signals from too many neighboring optodes.

The bulk material (body) of the phantom body structure should be selected to be weakly conductive so that that bulk material is able to effectively convey the electrical potentials generated by the optodes to the exterior surface of the phantom. For instance, salt ions or other charged particles may be incorporated within the bulk material to improve conductivity. The bulk material can be a liquid, gel, or even a solid. In one example, a ballistic gel or silicon rubber material may be employed. In another example, cells of a desired type may be mixed in agar to form the bulk material. And in a further example, the bulk material may be selected to be both weakly conductive and include particles capable of exhibiting the photo-voltaic effect. For instance, this may include a material in which very small silicon spheres or other nodules are patterned with electrodes, which can eliminate the need for discrete optodes coupled to the ends of the fibers. Here, phosphor could also be incorporated directly into the bulk material. The amount of conductivity throughout the structure, or in one or more localized regions within the structure, can be selected to mimic the tissue(s) under evaluation. By way of example, the brain, skull and scalp may all have different conductivities, and different bulk materials can be arranged to simulate such tissues. Furthermore, depending on the type of test and the particular use case, the phantom body structure may be shaped like the actual biological tissue(s), e.g., head, chest, etc., or it may have a general rectilinear, spherical or other shape.

In one scenario, a brain phantom configured as discussed above may include tens, hundreds or potentially thousands of optodes coupled to corresponding fibers. Each fiber may be less than 1 millimeter in diameter (e.g., 0.1-0.8 mm or more or less), and the spacing between the anode and cathode pads of the optode may be a fraction of a millimeter (e.g., 0.1-0.5 mm, or more or less). In addition, the fibers may not include any protective outer layer (cladding), which allows for them to have diameters smaller than 1 mm. The small size of the fibers and the photoreceiver structures thus enables the use of as many fiber/photoreceiver elements as desired.

Depending upon the electrical signals sought to be emulated, the optodes can be arranged in different 2D or 3D patterns within the phantom. The particular arrangement may vary depending on the biological counterpart and the type(s) of test to be conducted. For instance, the optodes may be sparsely arranged in one or more regions of the phantom while other regions are more densely populated. This can be done to simulate localized regions of activity, such as particular parts of the brain. By way of example only the frontal and temporal lobe regions of the phantom may be densely populated while the parietal and occipital lobe regions may be sparely populated. Thus, in one example the densely populated regions may have hundreds or thousands of optodes in total while the sparely populated regions may have 5-20 optodes in total, or more or less. In another example, the densely populated regions may have at least 10-50 optodes/cm³, while the sparsely populated regions may have on the order of 1-5 optodes/cm³, or more or less. In one scenario, the upper bound on the density of optodes is the neural density of the brain itself. In another scenario, the density of optodes may be limited by the physical size of each optode and/or the space available within the phantom body structure for the optical fibers.

The optodes may be held in place by some infrastructure (ing) so that their physical positions relative to the outer sensing surface of the phantom can be maintained. By way of example, a scaffold may be constructed by, e.g., 3D printing. Once constructed, the optodes are attached. Fibers are routed along the scaffolding and optically coupled to the optodes. After the fibers are arranged as desired, phantom bulk material can be poured into the structure, where it may be maintained as a liquid or cured until it forms into a gel or a solid.

As noted above, the sensors positioned at the surface of the phantom body structure are configured to detect electrical signals generated by one or more of the optically excited optodes within the phantom. The detected electrical signals output by the optodes can be a complex blend from different optode sources, such as can occur in an actual biological equivalent. By using optical fibers and other non-metallic components, the phantom need not include any magnetic materials. This will avoid interference with extremely sensitive sensors, such as MEG sensors. It also enables the reproducibility of signals, which can be particularly beneficial for receiver calibration and machine learning.

FIG. 5. illustrates an example 500 of a method of operating an optically excited biopotential phantom system, in which the system includes a phantom body structure having a set of optodes arranged therein. At block 502, one or more processors of a control module selects, in response to an input, a test or condition of interest from a set of biopotential scenarios. The input may be a selection by a user, or may occur in response to other operations of the system (e.g., as a result of startup calibration). At block 504, the one or more processors cause a signal modulator to modulate light emitted from one or more light sources according to the selected test or condition of interest from the set of biopotential scenarios. And at block 506, the modulated light causes selected optodes to operate in a photoresponsive mode so that each selected optode outputs an electrical signal as an excitation response to the modulated light. Here, the electrical signal output from each selected optode is detectable by one or more sensors disposed on a portion of the phantom body structure.

The method may further comprise detecting, by the one or more sensors disposed on the portion of the phantom body structure, at least some of the output electrical signals. It may also include evaluating, by a receiver device operatively coupled to the one or more sensors, the detected electrical signals. For instance, evaluating the detected electrical signals may include determining that the detected electrical signals correspond to a particular test or condition. In this case the method may also include comparing the particular test or condition to the selected test or condition of interest to determine whether the receiver device correctly evaluated the selected test or condition of interest.

The selected test or condition of interest may correspond to a brainwave pattern, a medical condition, a stimuli to be evaluated, a brain control interface command or other input. The method of operation may also include calibrating the set of optodes and/or the one or more sensors.

FIG. 6. illustrates an example 600 of a method of fabricating an optically excited biopotential phantom system. At block 602, the method provides a scaffolding corresponding to a biological structure or structure. At block 604, a set of optodes is coupled to a set of optical fibers. Each individual optode is optically engaged with a first end of a corresponding one of the optical fibers. Each optical fiber is configured to pass modulated light from a second end thereof to the first end. And each optode is configured to operate in a photoresponsive mode so that each optode outputs an electrical signal as an excitation response to the modulated light. At block 606, the method includes arranging the first ends of the set of optical fibers along the scaffolding so that the set of optodes are placed in a predetermined 2D or 3D pattern. And at block 608, the method includes forming a phantom body structure. The phantom body structure comprises a weakly conductive bulk material, for instance to enable the output electrical signals from the optodes to be received by sensors placed on or about the phantom body.

The bulk material may be a liquid, a gel or a solid. The weakly conductive bulk material may include salt ions or other charged particles.

Coupling the set of optodes to the set of optical fibers may include securing each corresponding optode to the first end of the corresponding optical fiber with an adhesive. For instance, the adhesive can be an optically clear adhesive. The adhesive can include a phosphorous material.

Forming the phantom body structure can include pouring the bulk material around the scaffolding. Alternatively, it can include inserting the scaffolding into the bulk material. And providing the scaffolding can include 3D printing the scaffolding.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements. The processes or other operations may be performed in a different order or simultaneously, unless expressly indicated otherwise herein.

1. An optically excited biopotential phantom system, the system comprising:
a phantom body structure including an electrically conductive bulk material, the phantom body structure being configured to represent one or more body tissues or structures;
a set of optical fibers, a first section of each optical fiber being received within the phantom body structure and a second section of each optical fiber extending from the phantom body structure, the first section having a first end of a respective one of the set of optical fibers and the second section having a second end of the respective one of the set of optical fibers; and
a set of optodes configured to operate in a photoresponsive mode, each one of the set of optodes being optically coupled to a corresponding one of the set of optical fibers;
wherein:
the second end of each one of the set of optical fibers is configured to receive an optical waveform from an optical modulation module and to pass the received optical waveform to the first end thereof;
the first end of each one of the set of optical fibers is configured to excite a corresponding one of the set of optodes optically coupled thereto based on the received optical waveform; and
each optode is configured to generate an electrical signal based on an excitation response to the received optical waveform, whereby the generated electrical signals from the set of optodes are detectable by one or more sensors disposed on a surface of the phantom body structure.

2. The system of claim 1, wherein the set of optodes are arranged in a 2D or a 3D pattern within the phantom.

3. The system of claim 2, wherein the 3D pattern includes at least one densely populated region and at least one sparsely populated region having fewer optodes than the at least one densely populated region.

4. The system of claim 1, wherein each optode of the set of optodes is either a photodiode or an LED configured to operate as a photoreceiver.

5. The system of claim 1, wherein each optode is optically coupled to the corresponding optical fiber with an adhesive.

6. The system of claim 5, wherein the adhesive is an optically clear adhesive.

7. The system of claim 5, wherein the adhesive includes a phosphorous material.

8. The system of claim 1, wherein the phantom body structure includes a scaffolding holding the set of optical fibers in a 3D pattern within the phantom.

9. The system of claim 1, wherein the bulk material is either a gel, a liquid or a solid.

10. The system of claim 1, wherein each one of the set of optical fibers is either a single mode fiber or a multi-mode fiber.

11. The system of claim 1, further comprising the optical modulation module.

12. The system of claim 1, further comprising the one or more sensors.

13. The system of claim 12, further comprising a receiver device operatively coupled to the one or more sensors.

14. The system of claim 12, wherein the one or more sensors are selected from the group consisting of electroencephalograph (EEG), electrocardiogram (ECG), electromyogram (EMG) or magnetoencephalography (MEG) sensors.

15. The system of claim 1, wherein the bulk material includes electrically conductive salt ions.

16. The system of claim 1, wherein the photoresponsive mode is a photovoltaic mode.

17. A method of operating an optically excited biopotential phantom system including a phantom body structure having a set of optodes arranged therein, the method comprising:
selecting, by one or more processors of a control module in response to an input, a test or condition of interest from a set of biopotential scenarios;
the one or more processors causing a signal modulator to modulate light emitted from one or more light sources according to the selected test or condition of interest from the set of biopotential scenarios; and
the modulated light causing selected ones of the set of optodes to operate in a photoresponsive mode so that each selected optode outputs an electrical signal as an excitation response to the modulated light, whereby the electrical signal output from each selected optode is detectable by one or more sensors disposed on a portion of the phantom body structure.

18. The method of claim 17, further comprising:
detecting, by the one or more sensors disposed on the portion of the phantom body structure, at least some of the output electrical signals; and
evaluating, by a receiver device operatively coupled to the one or more sensors, the detected electrical signals.

19. The method of claim 17, further comprising calibrating either the set of optodes or the one or more sensors.

20. A method of fabricating an optically excited biopotential phantom system, the method comprising:
providing a scaffolding corresponding to a biological structure or structure;
coupling a set of optodes to a set of optical fibers, each optode of the set being optically engaged with a first end of a corresponding one of the optical fibers, each optical fiber in the set being configured to pass modulated light from a second end thereof to the first end, and each optode of the set of optodes being configured to operate in a photoresponsive mode so that each optode outputs an electrical signal as an excitation response to the modulated light;
arranging the first ends of the set of optical fibers along the scaffolding so that the set of optodes are placed in a predetermined 2D or 3D pattern; and
forming a phantom body structure, the phantom body structure comprising a weakly conductive bulk material.
